(19) United States
(12) Patent Application Publication
(10) Pub. No.: US 2007/0048737 A1
(43) Pub. Date: Mar. 1, 2007

(54) Calcium phosphate ceramics and particles for in vivo and in vitro transfection

(76) Inventors: Nicole Francine Rouquet, Toulouse (FR); Patrick Pierre Frayssinet, Saiguede (FR)

Correspondence Address: FOLEY AND LARDNER LLP;SUITE 500, 3000 K STREET NW, WASHINGTON, DC, 20007, US

(21) Appl. No.: 10/540,854

(22) PCT Filed: Dec. 24, 2003
(86) PCT No.: PCT/FR03/03897

§ 371 Date: Sep. 1, 2006

(30) Foreign Application Priority Data
Dec. 27, 2002 (FR) 02/16785

(51) Int. Cl. C12Q 1/68; B05D 3/00

(52) U.S. Cl. 435/6; 427/2.11

(57) ABSTRACT

The invention relates to a method of modifying the surface of calcium phosphate ceramics and powders. The inventive method involves maturation in a culture medium, thereby causing epitaxial carbonated apatite growth at the surface of the aforementioned ceramics and powders. The invention also relates to the use of said modified ceramics and powders for in vitro and in vivo cell transfection and for cell culture in a three-dimensional network.

The present invention relates to a method for the transfection of DNA attached to the surface of calcium phosphate ceramics having specific characteristics. This method may comprise a step for preparing a material in a salt solution or a cell culture medium in order to improve the attachment of DNA and its availability for the transfection of cells. The invention also relates to the use of modified calcium phosphate ceramics and powders for the transfection of cells in vitro and in vivo and for the culture of transfected cells in a three-dimensional network.

The transfection of genes into eukaryotic cells is a key step in gene therapy. Several methods can be used with variable yields. They can be used in vitro or in vivo.

For the purposes of gene therapy, cells may be transfected in vitro and then reinjected into the body or directly into the organs or tissues in which they reside (Evans, C. H., Robbins, P. D., Possible orthopaedic applications of gene therapy, J Bone Joint Surg, 77-A, 7:1103-1114).

The various methods used for cell transfection are summarized in the table below:

| Method | Advantages | Disadvantages |
|---|---|---|
| DEAE-dextran | Simple | Transient expression |
| Calcium phosphate | Simple | Unusable for cells in |
| | | suspension |
| Liposomes | Simple | Relatively unproven |
| Microinjection | Efficient | Technically difficult |
| Electroporation | Good for nonadherent | No cotransfection |
| | cells | |
| Protoplast fusion | Good for nonadherent | Variable results |
| | cells | |
| Adenoviruses | High infectivity, | DNA integrated as |
| | unknown production, | episome, toxic, |
| | infects nondividing | production of viral |
| | cells, large variety | proteins |
| | of host cells | |
| Adeno-associated | Nonpathogenic, | Supports only short |
| viruses | stable expression, | genes, difficult to |
| | infects nondividing | produce, not highly |
| | cells, large variety | developed |
| | of host cells | |
| Herpessimplex | Infects nondividing | Toxic, transient |
| | cells, supports long | expression, not |
| | genes | highly developed |
| Retrovirus | Efficient | Cell type reduced by |
| infection | | tropism, low coding |
| | | capacity |
| Polycationic | Simple, localized | Transient expression |
| solids | transfection | |
| Satellite | Makes it possible to | Results unproven |
| chromosome | transfect long genes | |
| Others: polymers | | Low yields, variable |
| in the form of a | | results, uses in vivo |
| hydrogel, | | difficult, variable |
| polycationic | | biocompatibility |
| lipids, | | |
| polylysine, | | |
| polyornithine, | | |
| histones and | | |
| other chromosomal | | |
| proteins, | | |
| hydrogenated | | |
| polymers | | |

Approximately fifteen years after clinical trials of gene therapy started, the results have been disappointing overall for several reasons:

Regardless of the vectors used, adenoviruses, adeno-associated viruses (AAV), retroviruses or physicochemical formulations, the efficiency of gene transfer into the target cells has always been very low [A. Kahn. Dix ans de thérapie génique: deceptions et espoirs (Ten years of gene therapy: disappointments and hopes). Biofutur 202:16-21, 2000]. The duration of expression of the therapeutic transgenes is most often brief, limited to a few weeks, because of an immune reaction which causes the preferential elimination of the transduced cells, the intrinsic longevity thereof or the extinction of DNA sequences or promoters which direct the expression of the inserted genes (Orkin, S. H., Motulsky, A. G., report and recommendations of the panel to assess the NIH investment in research gene therapy.www.nih.gov/news/panelrep.html).

Finally, certain vectors have manifested a toxic effect. Accidents have occurred during the use of adenoviral vectors injected into the body which caused the death of patients in trials for treatment with ornithine transcarbamylase (Smaglik, P., Investigators ponders what went wrong after gene therapy death. The Scientist 13 [21]: 1 (1999).

Thus, it is evident from the analysis of all the clinical trials of gene therapy that the strategy of transfer of a gene would require vectors which are a lot more efficient, safer and capable of preferentially transfecting the cells on which a therapeutic effect is necessary (Orkin, S. H., Motulsky, A. G., report and recommendations of the panel to assess the NIH investment in research gene therapy. www.nih.gov/news/panelrep.html).

It is for this reason that polycationic polymeric vectors were developed. These vectors are solids and can adsorb DNA in various forms, in particular in the form of a plasmid. They have the characteristic feature of transfecting cells coming into contact with them with a variable yield. They have been used in vivo to transfect cells of the loose connective tissues involved in bone wound healing in order to accelerate it (S. Goldstein and J. Bonadio. in vivo gene transfer methods for wound healing. The Regent of the University of Michigan. Anonymous. U.S. Pat. No. (5,962,427):1-31, 1999. Gene Therapy. A61K 48/00. 514/44).

Coprecipitates of calcium phosphates and DNA have been used for many years to transfect cells in vitro (E. T. Schenborn and V. Goiffon. Calcium phosphate transfection of mammalian cultured cells. edited by M. J. Tymms, Totowa, N.J.:Humana Press Inc, 2000, p. 135-144; W. Song and D. K. Lahiri. Efficient transfection of DNA by mixing cells in suspension with calcium phosphate. Nucleic Acid Research 23 (17):3609-3611, 1995; Y.-W. Yang and J.-C. Yang. Calcium phosphate as a gene carrier: electron microscopy. Biomaterials 18:213-217, 1997).

They are obtained by pouring a calcium chloride solution into the medium in order to supersaturate it with calcium and to precipitate a calcium phosphate in which DNA molecules are embedded. These composite particles are then phagocytosed by the cells which integrate the plasmid in various ways and express the genes which are transported.

However, these coprecipitates have a major disadvantage. They are very difficult to use in vivo because it is difficult to obtain supersaturation in an open system. Moreover, they cannot allow localized transfections in space.

Calcium phosphate ceramics are materials obtained by sintering a slip containing particles of calcium phosphate in suspension. They are assemblages of grains linked by grain boundaries (Frayssinet, P., Fages, J., Bonel, G., Rouquet, N., Biotechnology, material sciences and bone repair. European Journal of Orthopaedic Surgery & Traumatology (1998) 8:17-25).

These materials exhibit a specific biocompatibility with the bone tissue, which makes them particularly useful as bone reconstruction materials or as vector for osteogenic cells (P., Frayssinet, J. L. Trouillet, N. Rouquet, E. Azimus, A. Autefage (1993), Osseointegration of macroporous calcium phosphate ceramics having a different chemical composition. Biomaterials, 14, 6: 423-429).

In the context of the invention, we have developed calcium phosphate ceramics and powders capable of transfecting cells both in vivo and in vitro, in particular mesenchymatous cells. The chemical composition of these ceramics may vary because several orthophosphoric acid salts can enter into their composition, in particular tricalcium phosphate, hydroxyapatite which is the synthetic phase closest to the mineral phase of the bone tissue, and octocalcium phosphate. These ceramics have another characteristic feature, they have surface properties which are highly variable according to various parameters such as, inter alia, the mode of synthesis of the powder, the baking temperature or the presence of various trace elements. These various factors influence in particular the surface charge, the zeta potential and the capacities for substitution in the mesh of the calcium phosphate. Calcium phosphate ceramics also have the characteristic feature of exhibiting epitaxial carbonated apatite growth at their surface once implanted in the body or immersed in a salt medium of comparable composition to the extracellular fluid (M. Heughebaert, R. Z. LeGeros, M. Gineste and A. Guilhem. Hydroxyapatite (HA) ceramics implanted in non-bone-forming sites. Physico-chemical characterization. J Biomed Mat Res 22:257-268, 1988). It is to these crystalline growths that the biocompatibility properties of these materials have been attributed.

The adsorption properties of calcium phosphates in relation to nucleic acids have been exploited in chromatography on HA columns to separate and purify DNA or certain RNAs. It is important to understand that given the same chemical composition, all the hydroxyapatite powders used in chromatography do not have the same nucleic acid separating power (A. Eon-Duval, Purification of plasmid DNA by hydroxyapatite chromatography, Abstract of 2nd conference on hydroxyapatite. San Francisco March 2001). The interactions between organic molecules and hydroxyapatite depends on the surface properties of this mineral (M. J. Gorbunoff, Protein chromatography on hydroxyapatite columns. Methods in Enzymology, vol 182, Academic Press Inc 1985: 329-339), which can vary from one batch to another.

It has been proved that the distribution of the charges at the surface of the solid and its hydration capacities have a great influence on the adsorption of organic molecules at its surface (Norde, W., Lyklema, J., (1991) Why proteins prefer interfaces. J Biomed Sci Polymer Edn 2, 183-202 (1991)). Likewise, the ionic strength and the pH of the solvent for organic molecules should be taken into account.

If the protein in solution and the solid have opposite charges, they attract each other. At least if the charge on the protein and that on the surface of the solid roughly balance. If the charges do not balance, that results in an accumulation of charges in the region of contact, causing a high electrostatic potential which is energetically not very favorable for an adsorption. A similar situation is observed when the surface of the solid and the organic molecule are of the same sign. However, in numerous cases, the adsorption may occur nevertheless in some cases by virtue of the incorporation of ions from the solution at the interface of the adsorbed layer which prevents the accumulation of charge.

The hydrophobicity has an influence on the adsorption because it participates in the distribution of the charges, in particular in organic molecules which have a tertiary and quaternary structure. The hydrophobicity of a surface (molecule or solid) can promote adsorption.

The distribution of the charges and the hydration capacities of apatites are advantageous properties because they may have a positive or negative surface charge and may be hydrophilic or hydrophobic. Furthermore, as substitutions in the mesh may be numerous, the functional groups at the surface can vary.

We have developed hydroxyapatite-based calcium phosphate powders capable of binding DNA in various forms and of delivering it to isolated cells or into the body for transfection purposes. These powders may be injected in suspension in a liquid or a gel. They may also be deposited using a spoon or serve as a transfecting vector for cells cultured in a three-dimensional network. They have specific physico-chemical properties in order to possess these transfection properties. A series of experiments were carried out which make it possible to judge the transfection of isolated or nonisolated cells with a plasmid carrying the gene for galactosidase which can be demonstrated by histochemistry. The powder is an embodiment which is particularly well suited to be able to transfect both isolated cells and tissues both in vitro and in vivo. These powders allow internalization of the DNA and its protection from intracytoplasmic nucleases and its transfer into the nucleus.

The mechanism involved in the attachment of DNA (an organic molecule having a negative charge) to the surface of hydroxyapatite particles may be:
An electrostatic adsorption when the material is positively charged.
A coprecipitation of the DNA molecules in the carbonated apatite layer appearing through epitaxial growth at the surface of these materials and resulting from complex dissolution/reprecipitation processes occurring at the surface in media supersaturated with calcium and phosphorus.
An ion exchange between the interfacial phase and the solution.

Once attached to the material, the DNA should penetrate into the cell. The composition and the surface characteristics are also important for the degradation of the material in a biological medium and the emission of transfecting particles. It is known that HA ceramics undergo degradation at the grain boundaries and that the carbonated apatite layer appearing at the surface of the material by epitaxial growth has a different solubility from the material itself.

On the other hand, all calcium phosphates cannot transfect cells. DCPD for example or certain embodiments of HA or TCP have shown their incapacity to do it. Their cytotoxicity is certainly responsible for that.

By contrast, the modification of the surface of the powders and the ceramics by maturation in a culture medium causing epitaxial carbonated apatite growth improves the labeling yield.

### DESCRIPTION

Thus, in a first aspect, the present invention relates to a method for creating a mineral-DNA composite, characterized in that it comprises a step consisting of an incubation in a salt or culture medium not saturated with calcium and phosphorus in the presence of the DNA molecule. This method makes it possible to obtain attachment of DNA to the surface of the ceramic by adsorption onto a ceramic surface modified by epitaxial growth or by coprecipitation at the surface of the material. These calcium phosphate particles are immersed in a salt medium or a culture medium of the cell culture media type commonly used in biotechnology, in particular DMEM, for a few minutes, for example 1, 5, 10 or 30 minutes at least to about 12, 24, 48 hours, a few days or more at a temperature ranging from 15 to 50° C., preferably about 37° C. The aim is to have the formation of a carbonated apatite layer at the surface before or during the bringing into contact with the plasmids.

In a particular embodiment, the method mentioned above is carried out before the bringing into contact with the nucleic acids, in particular plasmids. Alternatively, this step which causes epitaxial carbonated apatite growth at the surface of said powders and ceramics is carried out in a medium containing the nucleic acids. In this mode, the surface modification and the attachment of the nucleic acids are performed simultaneously. Preferably, the powders and ceramics are immersed in a DMEM culture medium for 48 hours at 37° C. before or simultaneously with the attachment of the nucleic acids.

In an additional aspect, the invention relates to a method for attaching DNA in plasmid form to the surface of calcium phosphate ceramic or powder, characterized in that it comprises a step a) consisting of a hydration of the calcium phosphate powder or calcium phosphate ceramic in a phosphate buffer solution not saturated with calcium and phosphate and a step b) consisting of an immersion of the products obtained in step a) in a phosphate buffer solution not saturated with calcium and phosphate containing a single- or double-stranded DNA for periods varying from a few minutes to several hours, c) producing calcium phosphate particles containing DNA molecules attached to its surface.

Preferably, the solution in step a) and b) comprises a 0.12 M phosphate buffer (pH 6.8). The immersion is carried out for at least 1, 5, 10 or 30 minutes up to about 12, 24 or 48 hours at a temperature ranging from 15 to 50° C., preferably about 37° C. In addition, the calcium phosphate particles are kept immersed in a culture medium of the cell culture media type, for about a few minutes to a few days, and at a temperature ranging from 15 to 50° C., preferably about 37° C.

Thus, in this method, the hydration preferably consists of an immersion of the calcium phosphate powder or of the calcium phosphate ceramic in a solution simulating the extracellular fluids intended to produce an epitaxial carbonated apatite growth at the surface of said powders and ceramics. In this regard, step b) is carried out by means of a medium simulating the extracellular fluids or a medium of the cell culture media type containing the nucleic acids, said medium being nondenaturing for the DNA and not saturated with calcium and phosphate. This medium causes epitaxial carbonated apatite growth at the surface of said powders and ceramics.

Steps a) and b) may be carried out simultaneously or successively. Thus, it is possible to carry out the invention with a solution containing a single- or double-stranded DNA for periods varying from a few minutes to several hours at about 37° C.

Advantageously, this method makes it possible to attach the DNA at physiological pH to calcium phosphate particles under conditions which are not denaturing for the DNA molecule. The ceramics may be porous or dense ceramics.

In another aspect, the invention relates to a method for transfecting isolated cells, cultured in a monolayer or in three dimensions, consisting of bringing the cells to be transfected into contact with the particles obtained by the method described above for periods of a few hours to a few weeks. This method can also be carried out to transfect cells contained in a cultured tissue fragment. The particles obtained which are mentioned above are particularly useful for the preparation of a medicament for transfecting in vivo cells contained in a tissue or in an organ.

In another aspect, the invention relates to the calcium phosphate ceramics and powders which can be obtained from the method described above, characterized in that they can support epitaxial carbonated apatite growth at their surface under nondenaturing conditions, in particular in a salt solution which is not saturated and nondenaturing for the biological macromolecules.

The invention also relates to these calcium phosphate ceramics and powders additionally comprising the nucleic acids attached to their surface.

These products are particularly efficient for the transfection of cells in vitro and in vivo.

Advantageously, the powders and ceramics obtained possess at least one of the properties described below before the surface modification:
nature of the charged groups at the surface: PO₄⁻, OH⁻, Ca⁺⁺
basic surface pH
negative electrokinetic potential
hydrophobic
particle size between 0-200 μm, in particular between 80-125 μm and 0-25 μm.

Preferably, the products of the invention comprise all the characteristics described above.

In addition, the calcium phosphate ceramics and powders mentioned above may contain a core composed of another polymeric, ceramic or metallic, preferably magnetic, material.

The invention also relates to the formed particles based on calcium phosphate powders described above, said particles being contained in a mineral or polymeric matrix, in particular in calcium sulfate or phosphate cements.

In another aspect, the invention relates to a ceramic coating for joint prostheses having the characteristics of the ceramic defined above.

The invention also relates to the use of said calcium phosphate ceramics and powders loaded with DNA at their surface as a support for cell culture, in particular for the culture in a three-dimensional network of cells transfected with the support and for the transfection of cells in vitro and in vivo.

The following examples are given by way of illustration. They constitute preferred embodiments of the invention.

### EXAMPLE 1

### Characteristic of the Powders Used

Type P15: spherical powder having a specific surface area of 0.62 mg²/g. They were calcined at 1180° C. and their particle size is between 80-125 μm.

Type P1: powder of any shape having a specific surface area of 56.84 m²/g, not calcined (crude), having a particle size of between 0-25 μm.

The study of the particle size of the powders used shows that the spherical powders (P15) have a well defined particle size range whereas those having any shape (P1) have much broader particle size ranges with a lot of fine particles. The zero charge pH varies with the powder calcination temperature. The zeta potential of the powder P1, measured in demineralized water, is −27.5 mV and the surface pH is 9.08.

Depending on the powder sintering temperature, the zero charge pH is variable but considerably less than the physiological pH. This means that regardless of the sintering temperature, the electrokinetic potential of the powders, at neutral pH, is negative.

Examination of the spherical powders by scanning microscopy shows that they consist of grains assembled through grain boundaries. Surface irregularities exist on some of the faces of the grains at high magnification.

Powder

| | P1 | P15 |
|---|---|---|
| Nature of the | PO₄⁻, OH⁻, Ca⁺⁺ | PO₄⁻, OH⁻, Ca⁺⁺ |
| charged groups | | |
| Electrokinetic | −27.5 | −35 |
| potential (mV) | | |
| Hydrophobicity | + | + |
| Surface pH | 9.8 | 7.8 |
| Particle size (μm) | 0-25 | 80-125 |
| Specific surface | 56.84 | 0.62 |
| area (m²/g) | | |
| Zero charge pH | | |
| Shape of the powder | angular | spherical |

### EXAMPLE 2

### Method of Attaching DNA to the Vector

The vector may be used in two different ways: Method A: It may be incubated directly with the plasmid in a phosphate buffer solution. It is then kept incubated therein for several hours while its surface is modified by epitaxial carbonated apatite growth. The attachment may then occur by coprecipitation at the surface of the material.

Method B: It may also be exposed to a salt solution for several days in order to modify the surface. Once the latter has reached equilibrium, the material is then placed in the solution containing the plasmid. The attachment of the DNA is assumed to then occur at the surface of the modified material.

Attachment of the Plasmid to the Surface of the Native Particles (Method A):

Double-stranded DNA has a marked affinity for HA when it is dissolved in low concentrations of phosphate buffer. They are eluted in higher concentrations of phosphate buffer. 1 m² of surface area of powder was deposited in Petri dishes, that is 1.61 g for type A and 0.017 g in type B.
Hydration of the HA powder (2 ml/g) in 10 ml of 0.12 M phosphate buffer at pH 6.8. Heating for 15 to 30 min at 100° C.
Allow to stand at room temperature and remove the buffer. Resuspend in 5 to 10 ml of 0.12 M phosphate buffer at pH 6.8 at 60° C., separate by decantation and resuspend in 5 ml of the same buffer at 60° C.
Add the nucleic acid sample in 1 ml of 0.12 M phosphate buffer at pH 6.8 at 40° C. (the elution of the double-stranded nucleic acids can be carried out by washing the HA 8 to 10 times with 0.5 ml of phosphate buffer (0.4 M)).

Attachment of the Plasmid to the Surface of the Particles Modified by Epitaxial Growth (Method B):
The particles were incubated at 37° C. in DMEM culture medium for 48 hours.
They are washed in a 0.12 M phosphate buffer solution at pH 6.8
The nucleic acid sample in 1 ml of 0.12 M phosphate buffer at pH 6.8 is added at 40° C.

### EXAMPLE 3

### Transfection of Cells In Vitro

Three lines were used:
Rabbit growth cartilage
Rabbit periosteum
Rat calvaria cells

They are obtained by digesting the collagenic matrix in a collagenase solution followed by centrifugation.

3.1 Material with Unmodified Surface

The quantity of powder (type B) was still the same: 10 mg.

During the transfection, the cells were not confluent. The cells were transfected at D0 and the first evaluation by histochemistry of the expression of galactosidase was made at D4, D21, D30.

At D4:

All the lines have labeling zones. In the wells transfected with particles, the labeled cells are grouped around particles although some were nevertheless distant from them. This distance can be explained by the fact that the particles emit debris with a high specific surface area. They can be seen under the microscope in the middle of groups of labeled cells. Growth cartilage cells: in absolute value, it is the series which was labeled the most.

At D21

As regards the cartilage cells, the number of transfected cells is high. The rat calvaria cells are highly positive.

At D30

The cells have a relative contact inhibition, they are practically in three dimensions and rounded. Most of the cells of the three groups are positive. The number of positive cells and the preceding growth rate appear to indicate that the plasmids are transmitted from one cell to another or alternatively that the release of DNA particles extends over time, the percentage of positive cells would have been very low in the opposite case. It is also possible that the release of transfecting particles is gradual. The cells preferentially labeled are those in contact with the particles.

Percentage of labeled cells as a function of the lines used:

| | | Conjugation | |
|---|---|---|---|
| Time (days) | calvaria | cartilage | periosteum |
| 4 | 15 | 32 | 27 |
| 21 | 39 | 42 | 35 |
| 30 | 65 | 71 | 60 |

3.2 Material with a Surface Modified by Epitaxial Growth:

From the early periods of culture, most of the cells are labeled.

3.2.1. Transfection on Either Side of a Semipermeable Membrane

The grains were deposited in contact with the cells or separated therefrom by a porous membrane (0.2 μm) made of polycarbonate separating them from the cellular lawn. Cellular labeling with galactosidase is evaluated by histochemistry on D4.

The cells in direct contact with the particles are labeled sporadically. The cells which are not in contact with the particles (separated by the membrane) are also labeled. Transfecting particles less than 0.2 μm in size therefore exist which pass across the pores of the polycarbonate membrane.

3.2.2 Transfection of Cells in a Three-Dimensional Network

The cell lines described above are suspended in the culture medium. The bed is placed at the bottom of a culture dish. The suspension serves to inoculate a bed of microbeads (1.5−2×10⁵ cells/0.05 g of beads) which are vectors for plasmids carrying the galactosidase gene. The bed is placed at the bottom of a culture dish. The cells are cultured for 10 to 15 days. The formation of a three-dimensional cellular layer bridging and agglomerating the beads is obtained. This layer also contains an abundant collagenic matrix.

On the observation date, the cells form a three-dimensional network bridging the various particles and assembling them. Optical microscopy reveals that the cells contained in the cluster of particles are labeled with galactosidase.

3.2.3 Transfection of Cells in Tissue Cultures

Material used for the labeling: Type A: spherical powder having a specific surface area of 0.62 m²/g. They were calcined at 1180° C. and their particle size is 80-125 μm. The quantity of powder is a few tens of particles per dish (P15).

A few beads were placed in contact with the bone fragments after having been incubated without pre-immersion (method A).

The bone fragments are obtained from femurs, tibias and calvaria of 3-day-old newborn rats. The bone pieces were cleaned to remove the accompanying soft tissues. The long bones were cut into three pieces: 2 epiphyses and the diaphysis. The calvarias were cut into small fragments with sides of 2 to 3 mm. These various fragments were deposited at the surface of a 3% agar gel in DMEM. The culture medium (DMEM+FCS) was then added so that the fragments show at the liquid-air interface.

The beads were kept in contact with the tissues for 2 to 30 days, on which date the galactosidase activity of the cells was demonstrated before preparing histological sections.

At 2 days of contact, sporadic labeling zones are identifiable. The labeling is made at a distance and in contact with HA beads. It also takes place in contact with these same beads.

At 30 days, the entire bone fragments changed to blue macroscopically (FIG. 1).

FIG. 1 represents a macrophotograph of a bone tissue culture in the presence of transfecting powder for 30 days. The bone fragment is completely blue because of the transfection of the cells with the galactosidase vector plasmid. Using reflection optical microscopy, it is not possible to see a zone which is not labeled. The beads are stuck in a matrix labeled by the reaction with galactosidase.

The sections of the various bone tissue samples cultured for 30 d show that the bone cells (osteoblasts, chondroblasts, perichondral cells, periosteal cells, osteoclasts) are labeled (FIG. 2 is a histological section of the same tissue showing that all the cells were transfected with galactosidase×30).

The hematopoietic cell lines are not labeled. It should be noted that:
all the bone cells are labeled
they are labeled regardless of the distance from the cells to the beads.
   3.2.4 Transfection In Vivo

A group of ten 4-week-old male NZW rabbits is selected randomly. These rabbits are divided into two groups: group A and group B. One rabbit from each group serves as control.

The operating zone is located on the left mandible behind the mandibular incisors. It should be noted that a preliminary study made it possible to select this site in which the bone is the most abundant. The P15 powder type was used. The DNA was attached by method A.

After the fitting of sterile fields and skin and mucosal disinfection, an intrabuccal vestibular incision is made using a bistoury. A strip of full thickness is inclined in order to reach the mandibular bony zone at the base of the incisors. A 3 mm trephine is used to systematize the bone diffraction. The bone defect made is of the order of 2 mm in depth. The bone flap is removed with the aid of a bone scissors. The biomaterial is sucked up with the aid of a 5 ml syringe and deposited in the bone defect such that it fills it. A slight pressure is used with a sterile gauze in order to keep the biomaterial in place. The repositioned flap is then sutured.

Two controls undergo a second controlateral operation without deposition of biomaterial.

The rabbits were sacrificed at 3 to 6 weeks. The mandibles were removed, fixed in ethanol and embedded in hydroxyethyl methacrylate. Sections 5 μm thick were made and the galactosidase activity demonstrated.

### At 3 Weeks:

In the Control Sites:

The histological sections show a spongy bone with few trabeculae whose pores are occupied by a very loose stromal tissue. Multinucleated cells of osteoclastic appearance exist at the surface of the trabeculae. These cells are all labeled by the galactosidase reaction. In the same manner, all the monocytes are also labeled. They are the only cells which are labeled.

In the Implanted Sites:

The sections passing through the calcium phosphate beads show that the beads are embedded in a relatively dense connective tissue with numerous multinucleated cells at their surface. All the fibroblastic or multinucleated cells are labeled with galactosidase.

When the sections become distant from the beads, fewer labeled cells exist; nevertheless, the tissue structures which were not disrupted by the surgical procedure give interesting information. The fibroblasts of the dental ligaments are labeled. Cell islets with a fibroblastic appearance exist which are labeled in the stromal tissue of the pores between the trabeculae. In some cases, it even appears that cells of the osteoblastic line are also labeled.

### 6 Weeks:

Macroscopically labeling exists around the HA grains. The sections show positive stromal cells, the cells of the dental ligaments and the odontoblasts express the gene.

1. A method for attaching DNA in plasmid form to the surface of calcium phosphate ceramic or powder, comprising a step a) consisting of a hydration of the calcium phosphate powder or calcium phosphate ceramic in a phosphate buffer solution not saturated with calcium and phosphate and a step b) consisting of an immersion of the products obtained in step a) in a phosphate buffer solution not saturated with calcium and phosphate containing a single- or double-stranded DNA for periods varying from a few minutes to several hours, c) producing calcium phosphate particles containing DNA molecules attached to its surface.

2. The method as claimed in claim 1, wherein the solution in step a) and b) comprises a 0.12 M phosphate buffer (pH 6.8).

3. The method as claimed in claim 1, wherein the immersion is carried out for at least 1, 5, 10 or 30 minutes up to about 12, 24 or 48 hours at a temperature ranging from 15 to 50° C., preferably about 37° C.

4. The method as claimed in claim 1, wherein the calcium phosphate particles are kept immersed in a culture medium of the cell culture media type.

5. The method as claimed in claim 4, wherein the calcium phosphate particles are immersed for about a few minutes to a few days.

6. The method as claimed in claim 4, wherein the calcium phosphate particles are immersed at a temperature ranging from 15 to 50° C., preferably about 37° C.

7. The method as claimed in claim 1, wherein step b) is carried out by means of a medium simulating the extracellular fluids or a medium of the cell culture media type containing the nucleic acids, said medium being nondenaturing for the DNA and not saturated with calcium and phosphate; this medium causing epitaxial carbonated apatite growth at the surface of said powders and ceramics.

8. The method as claimed in claim 1, wherein steps a) and b) are carried out simultaneously or successively.

9. The method as claimed in claim 7 to attach DNA under physiological pH conditions to calcium phosphate particles.

10. A method for transfecting isolated cells, cultured in a monolayer or in three dimensions, comprising bringing the cells to be transfected into contact with the particles obtained by the method as claimed in claim 1 for periods of a few hours to a few weeks.

11. A method for transfecting cells contained in a cultured tissue fragment consisting in bringing the cells to be transfected into contact with the particles obtained by the method as claimed in claim 1 for periods of a few hours to a few weeks.

12. A method of preparing a medicament for transfecting in vivo cells contained in a tissue or in an organ utilizing the particles obtained by the method as claimed in claim 1.

13. A calcium phosphate ceramic and powder which can be obtained from the method as claimed in claim 1, wherein epitaxial carbonated apatite growth at the surface of the calcium phosphate ceramic and powder under nondenaturing conditions is supported.

14. The calcium phosphate ceramic and powder as claimed in claim 13, additionally comprising nucleic acids attached to the surface of the calcium phosphate ceramic and powder.

15. The calcium phosphate ceramic and powder as claimed in claim 13 having at least one of the following properties:
nature of the charged groups at the surface: PO₄⁻, OH⁻, Ca⁺⁺
basic surface pH
negative electrokinetic potential
hydrophobic
particle size between 0-200 μm, in particular between 80-125 μm and 0-25 μm.

16. The calcium phosphate ceramic and powder as claimed in claim 13, comprising a core composed of another polymeric, ceramic or metallic, preferably magnetic, material.

17. A particle formed based on the calcium phosphate powder as claimed in claim 13, contained in a mineral or polymeric matrix, in particular in calcium phosphate or sulfate cements.

18. The calcium phosphate powders and ceramics as claimed in claim 13, for the transfection of cells in vitro.

19. The calcium phosphate ceramic and powder as claimed in claim 13, for the manufacture of a medicament for the transfection of cells in vivo.

20. The calcium phosphate ceramic and powder as in one claim 13, for the culture of transfected cells in three dimensions with formation of a cellular or extracellular matrix aggregating the particles.
